(19) United States
(12) Patent Application Publication
(10) Pub. No.: US 2009/0048492 A1
(43) Pub. Date: Feb. 19, 2009

(54) METHOD FOR MANAGING MULTIPLE PATIENT MONITORING SENSORS

(75) Inventors: Borje Rantala, Helsinki (FI); Matti Siltala, Helsinki (FI)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/839,238

(22) Filed: Aug. 15, 2007

(51) Int. Cl. A61B 5/00

(52) U.S. Cl. 600/300

(57) ABSTRACT

A method for associating a plurality of patient monitoring sensors with an appropriate patient is disclosed herein. The method includes estimating a sensor location for each of a plurality of sensors that are operatively connected to a patient, transmitting the sensor location estimate from each of the sensors to a wireless device such that the sensor location estimate does not pass through an on-patient hub before reaching the wireless device, and implementing the sensor location estimate to assign each of the sensors that are disposed within a predefined region to a single patient.

### BACKGROUND OF THE INVENTION

Patients within a hospital environment are generally monitored by system having a plurality of different sensors. The sensors may, for example, include a wristband sensor adapted to provide identity and location data, a pulse oximetry sensor, an electrocardiogram (ECG) sensor, and/or an electroencephalogram (EEG) sensor. Conventional patient monitoring systems also include an on-patient hub device adapted to receive data from the sensors, and to transmit such data to a hospital network. In order to manage data transfer in conventional patient monitoring systems, the specific type of data being transferred to the on-patient hub at any given time must be precisely identified and correlated with its appropriate sensor. It is therefore generally necessary to apply each sensor in a specific order and thereafter to remove each sensor in a specific order. One problem with such conventional patient monitoring systems is that the process of managing the transfer of data from the sensors to the on-patient hub device is complex and labor intensive. Another problem with such conventional patient monitoring systems is that the on-patient hub adds weight and cost to the system.

### BRIEF DESCRIPTION OF THE INVENTION

The above-mentioned shortcomings, disadvantages and problems are addressed herein which will be understood by reading and understanding the following specification.

In an embodiment, a method for associating a plurality of patient monitoring sensors with an appropriate patient includes estimating a sensor location for each of a plurality of sensors that are operatively connected to a patient, transmitting the sensor location estimate from each of the sensors to a wireless device such that the sensor location estimate does not pass through an on-patient hub before reaching the wireless device, and implementing the sensor location estimate to assign each of the sensors that are disposed within a predefined region to a single patient.

In another embodiment, a method for associating a plurality of patient monitoring system sensors with an appropriate patient includes acquiring sensor motion data for each of a plurality of sensors that are operatively connected to a patient, transmitting the sensor motion data from each of the sensors to a wireless device such that the sensor motion data does not pass through an on-patient hub before reaching the wireless device, implementing the sensor motion data to identify one or more common patterns of motion, and assigning each of the sensors having a common pattern of motion to a single patient.

In another embodiment, a method for associating a plurality of patient monitoring system sensors with an appropriate patient includes acquiring pulse data with each of a plurality of sensors that are operatively connected to a patient, transmitting the pulse data from each of the sensors to a wireless device such that the pulse data does not pass through an on-patient hub before reaching the wireless device, identifying each of the sensors that acquired pulse data having a common pulse data characteristic, and assigning each of the identified sensors to a single patient.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating a patient monitoring system connected to a patient;

FIG. 2 is a method for implementing the patient monitoring system of FIG. 1 in accordance with an embodiment;

FIG. 3 is a method for implementing the patient monitoring system of FIG. 1 in accordance with another embodiment; and

FIG. 4 is a method for implementing the patient monitoring system of FIG. 1 in accordance with yet another embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

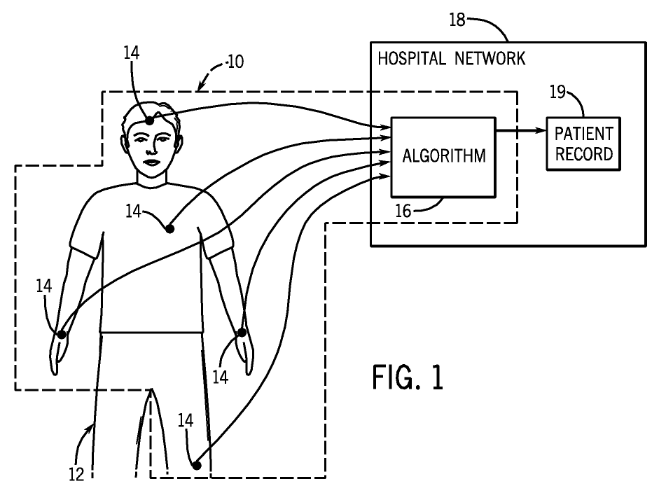

Referring to FIG. 1, a schematically illustrated patient monitoring system 10 is connected to a patient 12 in accordance with an exemplary embodiment. The patient monitoring system 10 includes one or more sensors 14 and an algorithm 16.

According to one embodiment, the sensors 14 each comprise a Wi-Fi wireless device compliant with the Institute of Electrical and Electronics Engineers (IEEE) 802.11 standard such that sensor data is wirelessly transmittable. The sensors 14 may include devices adapted to monitor patient location, pulse rate (e.g., a pulse oximetry sensor), cardiac electrical activity (e.g., an electrocardiogram or ECG sensor), electrical activity in a patient's brain (e.g., an electroencephalogram or EEG sensor), blood pressure, respiration, etc.

The sensors 14 may be referred to as “equal sensors” because there is no hierarchical relationship among the sensors 14 and none of the sensors 14 incorporate an on-patient hub. Advantageously, the absence of a hierarchical relationship enables the sensors to be implemented individually or in any combination with out regard to the order in which they are attached to or removed from a patient. Obviating the need for an on-patient hub reduces the overall expense and weight of the patient monitoring system 10.

The algorithm 16 may be stored on the hospital network 18. The algorithm 16 may, for example, be configured to perform one or more of the following operations: assign the sensors 14 to an appropriate patient; collect any sensor data from the sensors 14; and compile the sensor data into a patient record 19. For purposes of this disclosure, the assignment of sensors to an appropriate patient refers to the process of correlating or associating the individual sensors (and any data therefrom) with the patient to which the sensors are physically and/or operatively connected. A plurality of patient records 19 each containing data that pertains to a different patient may be stored on the hospital network 18.

It should be appreciated that a typical hospital environment may include multiple patient monitoring systems 10 that are each configured to monitor a different patients. It therefore becomes necessary to correlate the data from each of a plurality of different sensors with the patient to which the sensors are attached. Accordingly, a plurality of different methods for assigning the sensors 14 to an appropriate patient (i.e., the patient to which the sensors are attached) and for implementing the patient monitoring system 10 will hereinafter be described in detail.

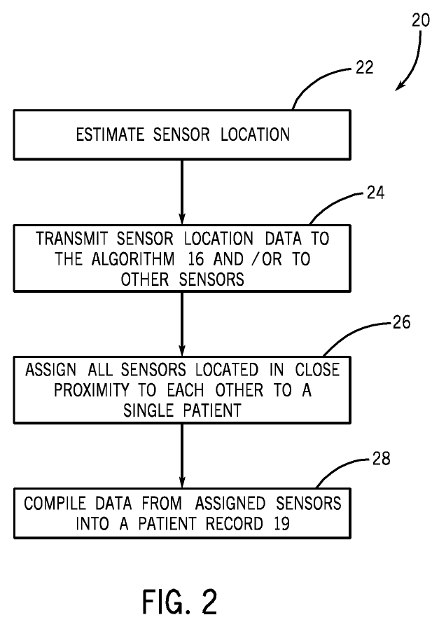

FIG. 2 is flow chart illustrating a method 20 in accordance with an embodiment. The method 20 can be included as a feature of the algorithm 16. The individual blocks of the flow chart shown in FIG. 2 represent steps that may be performed in accordance with the method 20.

Referring to FIGS. 1 and 2, at step 22 the method 20 estimates the location of each of the sensors 14. This step can be performed, for example, by incorporating a known locating device or system into each of the sensors 14.

At step 24, acquired location data is transmitted from each of the sensors 14. Step 24 can be performed in accordance with either of two distinct embodiments. According to a first embodiment, the sensors 14 are configured to transmit any acquired location data to the algorithm 16. Therefore, step 24 may be performed in accordance with the first embodiment by transmitting location data from each of the sensors 14 to the algorithm 16 such that the algorithm 16 can assign the sensors 14 to an appropriate patient. According to a second embodiment, each of the sensors 14 are configured to both transmit and receive any acquired location data. Therefore, step 24 may be performed in accordance with the second embodiment by transmitting location data among the sensors 14 so that the sensors 14 can assign themselves to an appropriate patient.

At step 26, the location data acquired by the sensors 14 is implemented to assign all the sensors 14 within a predefined region to a single patient. According to one embodiment, the predefined region may include all sensors located in sufficiently close proximity to each other. The requisite degree of proximity is selectable but may, for example, include any sensors located within four feet of each other so that a first sensor attached to a typical patient's head and a second sensor attached to the patient's leg would both be assigned to the same individual. If there is any ambiguity as to which patient a given sensor should be assigned at step 26, that sensor may be flagged and later manually assigned. In this manner, the likelihood of assigning sensor data to the wrong patient is minimized.

At step 28, sensor data collected from any of the sensors 14 that have been assigned is compiled in a convenient form such as, for example, the patient record 19. Patient records 19 for each of a plurality of different patients can then be stored on the hospital network 18.

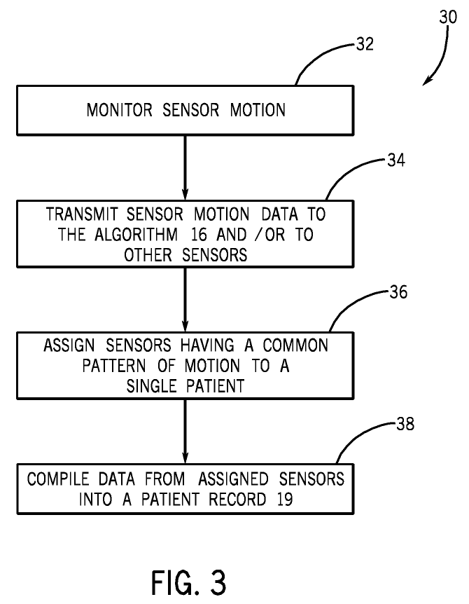

FIG. 3 is flow chart illustrating a method 30 in accordance with an embodiment. The method 30 can be included as a feature of the algorithm 16. The individual blocks of the flow chart shown in FIG. 3 represent steps that may be performed in accordance with the method 30.

Referring to FIGS. 1 and 3, at step 32 the method 30 monitors the motion of each of the sensors 14. This step can be performed, for example, by incorporating a known motion tracking device or system into each of the sensors 14.

At step 34, acquired motion data is transmitted from each of the sensors 14. Step 34 can be performed in accordance with either of two distinct embodiments. According to a first embodiment, the sensors 14 are configured to transmit any acquired motion data to the algorithm 16. Therefore, step 34 may be performed in accordance with the first embodiment by transmitting motion data from each of the sensors 14 to the algorithm 16 such that the algorithm 16 can assign the sensors 14 to an appropriate patient. According to a second embodiment, each of the sensors 14 are configured to both transmit and receive any acquired motion data. Therefore, step 34 may be performed in accordance with the second embodiment by transmitting motion data among the sensors 14 so that the sensors 14 can assign themselves to an appropriate patient.

At step 36, the motion data acquired by the sensors 14 is implemented to identify common patterns of motion and to assign all the sensors 14 having a common pattern of motion to a single patient. This step is predicated on the assumption that sensors attached to a single individual will share common motion traits. For example, there will be many instances when the sensors and the patient are all in motion, and further instances when the sensors and the patient are all at rest. Evaluating this sensor motion data may therefore be useful in the assignment of the sensors 14 to the appropriate patient. This sensor motion data may be used in combination with the previously described sensor location data in order to assign the sensors 14. If there is any ambiguity as to which patient a given sensor should be assigned at step 36, that sensor may be flagged and later manually assigned. In this manner, the likelihood of assigning sensor data to the wrong patient is minimized.

At step 38, sensor data collected from any of the sensors 14 that have been assigned is compiled in a convenient form such as, for example, the patient record 19. Patient records 19 for each of a plurality of different patients can then be stored on the hospital network 18.

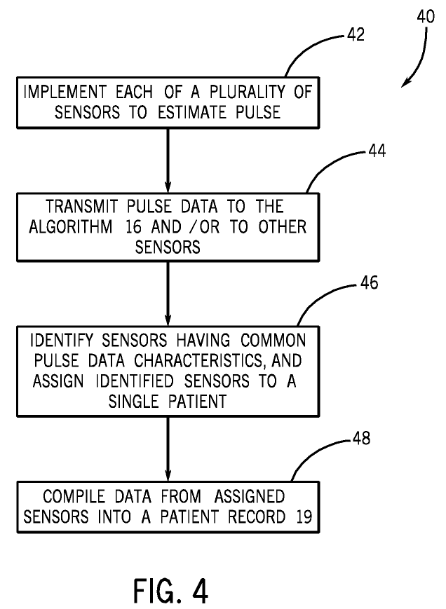

FIG. 4 is flow chart illustrating a method 40 in accordance with an embodiment. The method 40 can be included as a feature of the algorithm 16. The individual blocks of the flow chart shown in FIG. 4 represent steps that may be performed in accordance with the method 40.

Referring to FIGS. 1 and 4, at step 42 the method 40 acquires pulse data from each of the sensors. Many types of sensors such as pulse oximeter sensors and ECG sensors are adapted to acquire pulse data as their primary function. Additionally, it has been observed that EEG sensors attached to a patient's head can also be implemented to monitor a patient's pulse, and that conventional EEG sensors generally include a pulse signal component. This pulse signal component has typically been viewed as noise but may be useful for purposes of step 42. Other sensors that are not adapted to monitor pulse as a primary function and that do not provide a pulse signal component may require the addition of a separate pulse monitoring device.

At step 44, acquired pulse data is transmitted from each of the sensors 14. Step 44 can be performed in accordance with either of two distinct embodiments. According to a first embodiment, the sensors 14 are configured to transmit any acquired pulse data to the algorithm 16. Therefore, step 44 may be performed in accordance with the first embodiment by transmitting pulse data from each of the sensors 14 to the algorithm 16 such that the algorithm 16 can assign the sensors 14 to an appropriate patient. According to a second embodiment, each of the sensors 14 are configured to both transmit and receive any acquired pulse data. Therefore, step 44 may be performed in accordance with the second embodiment by transmitting pulse data among the sensors 14 so that the sensors 14 can assign themselves to an appropriate patient.

At step 46, the pulse data acquired by the sensors 14 is implemented to identify each of the sensors 14 having similar pulse data characteristics, and to assign all the sensors 14 identified as having similar pulse data characteristics to a single patient. This step is predicated on the assumption that multiple patients are unlikely to share pulse characteristics such as pulse rate, pulse phase, and pulse variation. Evaluating this pulse data may therefore be useful in the assignment of the sensors 14 to the appropriate patient. This pulse data may be used in combination with the previously described sensor pulse data and/or the previously described sensor location data in order to assign the sensors 14. If there is any ambiguity as to which patient a given sensor should be assigned at step 46, that sensor may be flagged and later manually assigned. In this manner, the likelihood of assigning sensor data to the wrong patient is minimized.

At step 48, sensor data collected from any of the sensors 14 that have been assigned is compiled in a convenient form such as, for example, the patient record 19. Patient records 19 for each of a plurality of different patients can then be stored on the hospital network 18.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

We claim:

1. A method for associating a plurality of patient monitoring sensors with an appropriate patient comprising:
estimating a sensor location for each of a plurality of sensors that are operatively connected to a patient;
transmitting the sensor location estimate from each of said plurality of sensors to a wireless device such that the sensor location estimate does not pass through an on-patient hub before reaching the wireless device; and
implementing the sensor location estimate to assign each of said plurality of sensors that are disposed within a predefined region to a single patient.

2. The method of claim 1, wherein said estimating a sensor location includes estimating a sensor location for each of a plurality of equal sensors.

3. The method of claim 1, wherein said transmitting the sensor location estimate to a wireless device includes transmitting the sensor location estimate to a hospital network.

4. The method of claim 1, wherein said transmitting the sensor location estimate to a wireless device includes transmitting the sensor location estimate to one or more of said plurality of sensors.

5. The method of claim 1, wherein said implementing the sensor location estimate includes implementing the sensor location estimate to assign each of said plurality of sensors that are disposed in sufficiently close proximity to each other.

6. The method of claim 1, further comprising manually assigning any of said plurality of sensors that are not assigned based on the sensor location estimate.

7. A method for associating a plurality of patient monitoring system sensors with an appropriate patient comprising:
acquiring sensor motion data for each of a plurality of sensors that are operatively connected to a patient;
transmitting the sensor motion data from each of said plurality of sensors to a wireless device such that the sensor motion data does not pass through an on-patient hub before reaching the wireless device;
implementing the sensor motion data to identify one or more common patterns of motion; and
assigning each of said plurality of sensors having a common pattern of motion to a single patient.

8. The method of claim 7, wherein said acquiring sensor motion data includes acquiring sensor motion data for each of a plurality of equal sensors.

9. The method of claim 7, wherein said transmitting the sensor motion data to a wireless device includes transmitting the sensor motion data to a hospital network.

10. The method of claim 7, wherein said transmitting the sensor motion data to a wireless device includes transmitting the sensor motion data to one or more of said plurality of sensors.

11. The method of claim 7, further comprising manually assigning any of said plurality of sensors that are not assigned based on the sensor motion data.

12. A method for associating a plurality of patient monitoring system sensors with an appropriate patient comprising:
acquiring pulse data with each of a plurality of sensors that are operatively connected to a patient;
transmitting the pulse data from each of said plurality of sensors to a wireless device such that the pulse data does not pass through an on-patient hub before reaching the wireless device;
identifying each of said plurality of sensors that acquired pulse data having a common pulse data characteristic; and
assigning each of said identified sensors to a single patient.

13. The method of claim 12, wherein said acquiring pulse data includes acquiring pulse data with each of a plurality of equal sensors.

14. The method of claim 12, wherein said transmitting the pulse data to a wireless device includes transmitting the pulse data to a hospital network.

15. The method of claim 12, wherein said transmitting the pulse data to a wireless device includes transmitting the pulse data to one or more of said plurality of sensors.

16. The method of claim 12, wherein said identifying each of said plurality of sensors having a common pulse data characteristic includes identifying each of said plurality of sensors having a common pulse rate, a common pulse phase, and/or a common pulse variation.

17. The method of claim 12, further comprising manually assigning any of said plurality of sensors that are not assigned based on the pulse data.
